(19) United States
(12) Patent Application Publication
(10) Pub. No.: US 2017/0035933 A1
(43) Pub. Date: Feb. 9, 2017

(54) ARTIFICIAL BIOMATERIAL COMPRISING COPPER-BASED COMPOUND

(71) Applicant: BS SUPPORT CO., LTD., Suwon-si Gyeonggi-do (KR)

(72) Inventor: Seung Woo BAEK, Anyang-si (KR)

(21) Appl. No.: 15/035,010

(22) PCT Filed: Nov. 6, 2014
(86) PCT No.: PCT/KR2014/010595

§ 371 Date: May 6, 2016

(30) Foreign Application Priority Data
Apr. 28, 2014 (KR) 10-2014-0050489

(51) Int. Cl. A61L 27/30; A61L 27/10; A61L 27/16; A61L 27/44; A61L 27/06; A61L 27/12

(52) U.S. Cl. CPC A61L 27/306; A61L 27/06; A61L 27/12; A61L 27/16; A61L 27/446; A61L 27/10; A61L 2420/02; A61L 2300/404; A61L 2300/102; A61L 2430/24

(57) ABSTRACT

An artificial biomaterial comprises: a biocompatible matrix suitable for insertion into the human body; and either a coating layer comprising the copper-based compound, formed on the surface of the matrix, or the copper-based compound dispersed in the matrix to form a bulk material, wherein the copper-based compound has a chemical structure of CuₓM_{y}, wherein M is any one selected from among elements belonging to groups 15 to 17 of the periodic table, and x/y=0.8-1.5.

### BACKGROUND OF THE INVENTION

Field of the Invention

The present invention relates to a biomaterial comprising a copper-based compound, and more particularly, to an artificial biomaterial that has increased antimicrobial activity by virtue of a conductive copper-based compound.

Description of the Prior Art

Artificial biomaterials refer to materials that are used in contact with living tissues in addition to human normal skins. Increases in persons with disabilities caused by various accidents and in aging populations have led to an increase in the use of artificial biomaterials that are inserted to the human body to replace injured parts or assist in the treatment of organs such as hearts. Currently, artificial biomaterials, including artificial bones, artificial joints, artificial teeth and stents, are being used in a very wide range of applications. It appears that many parts of the human body may be replaced with artificial biomaterials in future. Matrix materials that are used for artificial biomaterials can be largely divided into metal materials, polymer materials, ceramic materials, and composite materials. Artificial biomaterials may be at risk of being infected with pathogens during their transplantation into the human body and surgery, because they have poor antimicrobial activity. Pathogens easily form colonies on the surface of biomaterials to cause serious contamination problems.

In the prior art, silver (Ag) and silver salts that release silver ions have been used to impart antimicrobial activity to artificial biomaterials. Silver (Ag) has high toxicity against bacteria even at its very low concentration, and pathogens are less likely to develop resistance to silver. U.S. Pat. No. 3,800,087, German Patent No. 4328999, and Korean Patent No. 10-0987728 disclose methods of coating silver on the surface of biomaterials. In addition, WO 01/09229A1, WO 2004/024205 A1, EP 0 711 113 A and the literature [Muenstedt et al., Advanced Engineering Materials 2000, 2(6), pages 380 to 386] disclose methods of incorporating nanocrystalline silver into thermoplastic polyurethane.

However, when silver (Ag) is coated on or incorporated into biomaterials, a process for increasing the adhesion of silver (Ag) is very complex, and only a small amount of silver (Ag) of the silver (Ag) added exhibits antimicrobial activity. Although silver (Ag) is known to have excellent antimicrobial activity, the practical use of the silver (Ag) is difficult due to many limitations. Although silver (Ag) has high antimicrobial activity and is convenient to use, it is excessively costly. In an attempt to solve this problem, a salt of silver (Ag) was also used for antimicrobial coating. However, a salt of silver may be present as an anion that may have toxicity in a certain environment, unlike silver (Ag). Because some silver salts such as silver nitrate are very easily dissolved in water, the silver ions thereof can be transferred too quickly from a surface coating to the surrounding portion, and because other silver salts such as silver chloride have poor solubility, they can form a silver solution too slowly.

### SUMMARY OF THE INVENTION

It is an object of the present invention to provide an artificial biomaterial comprising a copper-based compound, which is relatively low-priced, easily processed, non-toxic, and has excellent antimicrobial activity.

To achieve the above object, the present invention provides an artificial biomaterial comprising a copper-based compound, the artificial biomaterial comprising: a biocompatible matrix suitable for insertion into the human body; and either a coating layer comprising the copper-based compound, formed on the surface of the matrix, or the copper-based compound dispersed in the matrix to form a bulk material. Herein, the copper-based compound has a chemical structure of CuₓM_{y}, wherein M is any one selected from among elements belonging to groups 15 to 17 of the periodic table, and x/y=0.8-1.5.

In the artificial biomaterial of the present invention, M may be any one selected from among S, F and Cl, and the copper-based compound is preferably copper sulfide. The content of the compound may be more than 0 wt % but not more than 50 wt %, preferably 0.01-30 wt %, based on the weight of the bulk material.

In the artificial biomaterial according to a preferred embodiment of the present invention, the coating layer may be formed by any one method selected from among wet application, vapor deposition, and coating. If the coating layer is formed by wet application, a coating solution for forming the coating layer may contain the compound in an amount of more than 0 wt % but not more than 50 wt %, preferably 0.01-30 wt %.

In the artificial biomaterial according to a preferred embodiment of the present invention, the biocompatible matrix may be any one selected from among a metal material, a ceramic material, a polymer material, and a composite material comprising at least one of these materials. The artificial biomaterial may comprise the compound coated on the surface of any one matrix selected from among the metal material, the ceramic material and the composite material. The artificial biomaterial may comprise the compound coated on the surface of any one matrix selected from among the polymer material and the composite material or may comprise the compound dispersed in the selected matrix.

In a preferred embodiment of the present invention, the artificial biomaterial comprising the compound dispersed therein may comprise fine particles of at least one metal selected from among chromium, manganese, iron, cobalt, nickel and zinc in an amount of 0.1-2 wt % based on the total weight of the biomaterial. Before the compound is coated on the compatible matrix, a coating solution containing 0.01-1.0 wt % of fine colloidal particles of a transition metal and 0.01-2.0 wt % of an emulsion of at least one selected from among water-soluble polyester, water-soluble urethane and water-soluble acrylic resin may be applied.

In a preferred embodiment of the present invention, the artificial biomaterial may be applied to an artificial joint, and may comprise a layer of the compound plated on the surface of a matrix for the artificial joint. The plating may be electroless plating or electroplating. The plating is preferably performed by performing electroless plating followed by electroplating. The plating layer may have a thickness of 0.01-5.0 μm.

In the artificial joint that is the artificial biomaterial of the present invention, before the plating layer is formed, the surface of the artificial joint may be treated with a conductive polymer emulsion solution containing a transition metal. Herein, an aqueous dispersion containing 0.01-1.0 wt % of fine colloidal particles of a transition metal and 0.01-2.0 wt % of an emulsion of at least one selected from water-soluble polyester, water-soluble urethane and water-soluble acrylic resin may be applied to the surface of the artificial joint. Herein, the solid content of the aqueous dispersion containing the transition metal is preferably controlled such that the solids of the aqueous dispersion remain on the surface of the artificial joint in an amount of 0.001-0.1 g/m².

### BRIEF DESCRIPTION OF THE DRAWINGS

FIG. 1 is an XRD graph showing the crystal structure of copper sulfide prepared according to the present invention.

FIGS. 2A and 2B are photographs of the top and cross-section of a copper sulfide-coated artificial biomaterial prepared according to the present invention.

### DETAILED DESCRIPTION OF THE INVENTION

Hereinafter, preferred embodiments of the present invention will be described with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art.

Embodiments of the present invention provide an artificial biomaterial comprising a copper sulfide-based compound, which is relatively low-priced, easily processed, non-toxic, and has excellent antimicrobial activity. Artificial biomaterials are materials that are inserted into the human body to replace injured parts or assist in the treatment of organs. Currently, artificial biomaterials include artificial bones, artificial joints, artificial teeth, stents, etc., and it appears that the application of such artificial biomaterials will be expanded in future. Methods of imparting antimicrobial activity to such artificial biomaterials include a dispersion method in which a copper sulfide compound is dispersed in a biocompatible matrix, and a coating method in which the copper sulfide compound is coated on the surface of the biocompatible matrix. The embodiments of the present invention will be described in detail in terms of each of the dispersion method and the coating method. In addition, a process of plating a copper-based compound on an artificial joint that is an artificial biomaterial will be described in detail.

Biocompatible matrixes for artificial biomaterials can be largely divided into metal materials, polymer materials, ceramic materials, ceramic materials, and composite materials. The metal materials include stainless steel, chromium-cobalt based alloys, titanium-based alloys, etc., and the ceramic materials include calcium oxide-silica activated glass and crystallized glass, calcium phosphate compounds containing apatite that is the inorganic component of bone, etc. Among the polymer materials, thermoplastic resins, particularly biodegradable polymers are increasingly used, and the composite material is a material obtained by mixing two or more, selected among metal, polymer and ceramic materials, with each other at a certain ratio.

Polymer resins, that is, both thermoplastic resins and thermosetting resins, may be used in the present invention. Among these resins, thermoplastic resins advantageous in terms of molding are preferably used. Specific examples of thermoplastic resins that may be used in the present invention include polyethylene terephthalate, polylactic acid, polyethylene, polypropylene, polycarbonate, polymethylmethacrylate, polyvinyl chloride, polyurethane, silicone, etc. Thermosetting resin that may be used in the present invention is preferably epoxy resin or the like. Polyurethane is more preferably used in the present invention, it is flexible, non-toxic, and has good chemical resistance.

The copper-based compound that is used in the embodiment of the present invention is preferably copper sulfide (CuS). In the present invention, copper sulfide was synthesized by reacting copper sulfate (CuSO₄) with a salt, selected from among sulfide salts, fluoride salts and chloride salts, at a molar ratio of 1:1 in an aqueous solution at a temperature of 50 to 80° C. Herein, the synthesized copper sulfide had a chemical structure CuₓS_{y}, and the synthesis was performed such that the ratio of x/y would satisfy 0.8-1.5. Examples of sulfide salts that may be used in the present invention include sodium sulfide, iron sulfide, potassium sulfide, zinc sulfide, etc., and examples of fluoride salts that may be used in the present invention include sodium fluoride, iron fluoride, potassium fluoride, zinc fluoride, etc. In addition, examples of chloride salts that may be used in the present invention include sodium chloride, iron chloride, potassium chloride, zinc chloride, etc. Herein, copper sulfide synthesized using sodium sulfide and copper sulfate had the best antimicrobial activity.

If the reaction temperature is lower than 50° C., the reactivity between copper sulfate and a salt in the synthesis of copper-based nanoparticles will be low, but the synthesized copper-based nanoparticles will have good antimicrobial activity. However, the yield of production of copper sulfide will be low. If the reaction temperature is higher than 80° C., the reaction rate will excessively increase, and thus the density of crystals on the surface of copper sulfide will increase and the concentration of copper will increase, resulting in a decrease in the antimicrobial activity of copper sulfide. In addition, if the ratio of x/y in the copper-based nanoparticles is less than 0.8, the concentration of sulfur will excessively increase, and thus the nanoparticles will have good antimicrobial activity. However, the chemical stability of copper sulfide will decrease. If the ratio of x/y is more than 1.5, the concentration of copper will increase, resulting in a decrease in the antimicrobial activity.

Copper sulfide is decomposed at a temperature of 400° C. or higher so that sulfur (S) is separated from the copper sulfide (CuS). As sulfur (S) is separated, pores are formed in the copper sulfide (CuS). Biomaterials such as metal materials or ceramic materials are generally formed into artificial biomaterials by casting or sintering. Because casting or sintering is performed at a temperature of at least 400° C. at which sulfur (S) is separated from copper sulfide (CuS), the method of dispersing copper sulfide is not appropriate. An artificial biomaterial comprising a metal material or a ceramic material is preferably prepared by coating copper sulfide on the surface of a biocompatible material using the coating method. Dispersion of fine copper sulfide particles in polymer materials or some composite materials can be achieved by a compounding method at relatively low temperatures. Thus, both the dispersion method and the coating method may be used in the present invention depending on circumstances.

For convenience of explanation, biocompatible materials will be divided into a first material to which the coating method is properly applied, a second material to which the dispersion method may be applied, and a third material to which both the dispersion method and the coating method may be applied. The embodiments of the present invention will be described with a focus on the third material to which both the dispersion method and the coating method may be applied. The coating method for the third material may be applied to the first and second materials, and the coating method for the third material may be applied to the first and second material. Hereinafter, the present invention will be described with a focus on copper sulfide among the above-described compounds.

Artificial Biomaterial Having Fine Copper Sulfide Particles Dispersed Therein

As the dispersion method, a mixing method or a compounding method may be used. Particularly, the compounding method is preferably used. A biomaterial may be shaped by extrusion, injection, cutting or the like. The compounding method is used to increase the dispersion of fine copper sulfide particles in polymer resin. The compounding is performed at a barrel temperature that is 30-50° C. higher than the melting temperature of the resin. The compounding is performed in a compounding machine equipped with a biaxial unidirectional screw showing better dispersibility than a uniaxial screw. The compounding machine preferably has an L/D ratio ranging from 30 to 40. The compounded resin is stored as chips in a bunker. Where the compounded material is extruded, the extrusion is performed at a temperature that is 30-50° C. higher than the melting temperature of the polymer resin used. Next, molding, first cooling, annealing and second cooling steps are performed, thereby providing an artificial biomaterial having a desired shape. The content of the copper sulfide is preferably more than 0 wt % but not more than 50 wt %, preferably 0.01-30 wt %, based on the bulk-type biomaterial.

However, if the fine copper sulfide particles according to the embodiment of the present invention are compounded with the polymer resin to prepare an artificial biomaterial, the dispersibility of the copper sulfide particles will decrease. For this reason, the extrusion pressure in the extrusion process may increase. In order to prevent the extrusion pressure from increasing, in the embodiment of the present invention, fine particles of at least one metal selected from the group consisting of chromium, manganese, iron, cobalt, nickel and zinc, which are transition metals belonging to period 4 of the periodic table, may be added in an amount of 0.1-2 wt % based on the weight of the biocompatible material. If the transition metal is mixed with the copper-based material, it shows excellent dispersibility and excellent antimicrobial activity compared to a typical metal such as Al.

To reduce the extrusion pressure, the mean particle diameter of the fine metal particles is smaller than that of fine particles of the copper-based compound. If the fine metal particles are added to thermoplastic resin in an amount smaller than 0.1 wt % or larger than 2 wt %, the extrusion pressure will increase rather than decrease. As described above, the fine metal particles are added to control the extrusion pressure, and the required antimicrobial activity of the artificial biomaterial can be obtained only by the copper-based compound. Thus, according to the present invention, the artificial biomaterial can be prepared without having to use the fine metal particles. Herein, as the fine metal particles added, those that do not impair the required antimicrobial activity of the artificial biomaterial of the present invention are selected.

Artificial Biomaterial Having Fine Copper Sulfide Particles Coated Thereon

Coating of copper sulfide on the surface of the biocompatible material according to the present invention may be performed by various methods, including wet application, plating, deposition and the like. The wet application method shows low adhesive strength compared to the plating or deposition method, but has advantages in that it is simple and inexpensive. Copper sulfide powder may be added to a solvent containing IPA, toluene, benzene, a binder or the like and be sufficiently dispersed to make a coating solution, and the coating solution may be coated on the surface of the biocompatible material by a method such as dip coating or spray The concentration of copper sulfide is determined considering dispersibility and a thickening phenomenon. If a dispersing agent is used, a high-concentration coating solution can be prepared. The content of the copper sulfide is preferably more than 0 wt % but not more than 50 wt %, more preferably 0.01-30 wt %.

The coating thickness of copper sulfide is preferably about 300-600 Å, and can be controlled by repeating coating or controlling the viscosity of the coating solution. The coated biomaterial is dried. Preferably, it is subjected to step 1 of drying at low temperature and step 2 of sintering. Step 1 is a step of slowly removing water and the solvent from the coating solution, and is preferably performed at a temperature of 90 to 100° C. for 1-2 hours. Step 2 is a step of increasing the bonding between the copper sulfide particles. Because copper sulfide is likely to be decomposed at a temperature of 400° C. or higher, the sintering is preferably performed at a temperature of 200 to 300° C. for 1-2 hours. If the drying is performed at an excessively high temperature for an excessively long time, the coating layer will be cracked to make the appearance poor, and the sulfur component will be separated to significantly reduce the antimicrobial activity. Particularly in the case of spray coating, a coating solution prepared using a supercritical fluid such as carbon dioxide is preferably used. The supercritical fluid can eliminate the toxicity of organic solvent and shorten the drying time.

For deposition, copper sulfide is first synthesized to prepare a vacuum deposition target. To the surface of the biocompatible material, an aqueous dispersion containing 0.01-1.0 wt % of colloidal transition metal particles and 0.01-2.0 wt % of an emulsion of at least one selected from water-soluble polyester, water-soluble urethane and water-soluble acrylic resin is applied. The aqueous dispersion can increase the strength of the deposited layer. The solid content of the aqueous dispersion is controlled such that the solids of the aqueous dispersion remain on the surface of the biocompatible material in an amount of 0.001-0.1 g/m². For vapor deposition, heating is performed under a vacuum of 10⁻⁵-10⁻³ Torr such that the vapor deposition of the metal (10⁻²-10⁻¹ Torr) is maintained, thereby depositing copper sulfide on the surface of the biocompatible material to a thickness of 300-600 Å. The strength of the deposited layer is preferably maintained at at least 60 g/25 mm.

The plating method has disadvantages in that it is difficult to carry out and is expensive, compared to the deposition or wet application method. However, it is suitable for the preparation of an artificial biomaterial that is repeatedly used for a long period of time because of its excellent durability. To increase the plating strength, the surface of the compatible material is preferably treated with a conductive polymer emulsion solution containing a transition metal, because it is plated. To the surface of the biocompatible material, an aqueous dispersion containing 0.01-1.0 wt % of colloidal transition metal particles and 0.01-2.0 wt % of an emulsion of at least one selected from water-soluble polyester, water-soluble urethane and water-soluble acrylic resin is applied. The solid content of the aqueous dispersion is controlled such that the solids of the aqueous dispersion remain on the surface of the biocompatible material in an amount of 0.001-0.1 g/m². The plating process may be performed by introducing copper sulfide into a plating bath, ionizing the introduced copper sulfide, and then performing electroplating or electroless plating. For example, in the plating method, a copper salt and a sulfur compound may be added to a plating solution, and copper sulfide may be deposited on the surface of the biocompatible material using a reducing agent so that it can adhere to the surface. The thickness of copper sulfide plated on the biocompatible material is preferably 0.01-5.0 μm.

In an example of the present invention, dip coating was used. Specifically, copper sulfide was added to a solvent such as isopropyl alcohol (IPA) in a predetermined amount, and stirred at room temperature for several hours to prepare a coating solution having excellent dispersibility. Then, a biocompatible material was dip-coated with the coating solution. The coated biocompatible material was dried at several tens of ° C. for several hours, and then annealed at 400° C. or below for several tens of minutes. To provide a biomaterial having excellent antimicrobial activity, coating was repeated in the same manner so that the copper sulfide could be sufficiently coated on the surface of the biocompatible material.

Hereinafter, the present invention will be described in further detail with reference to examples. It is to be understood, however, that these examples are for illustrative purposes only and are not intended to limit the scope of the present invention. The performance of artificial biomaterials prepared in the examples and comparative examples of the present invention was evaluated in the following manner.

(1) Antimicrobial Activity

Escherichia coli (ATCC 25922) was used as a test strain. A sample was brought into contact with the test strain, and then incubated at 25° C. for 24 hours. Then, the number of the bacterial cells was counted, thereby evaluating the antimicrobial activity of the sample.

(2) Extrusion Pressure

The dispersibility of copper sulfide and fine metal particles in polymer resin was evaluated by a change in extrusion pressure applied to a filter. When 30 kg/hr of resin was extruded using a pilot extruder, a change in filter pressure applied to a 350-mesh filter per certain time, {[kg/(mm²×h)]=ΔP/h}, was measured. It was determined that, as the change in the filter pressure was smaller, the dispersibility of copper sulfide and fine metal particles was better.

### Example 1

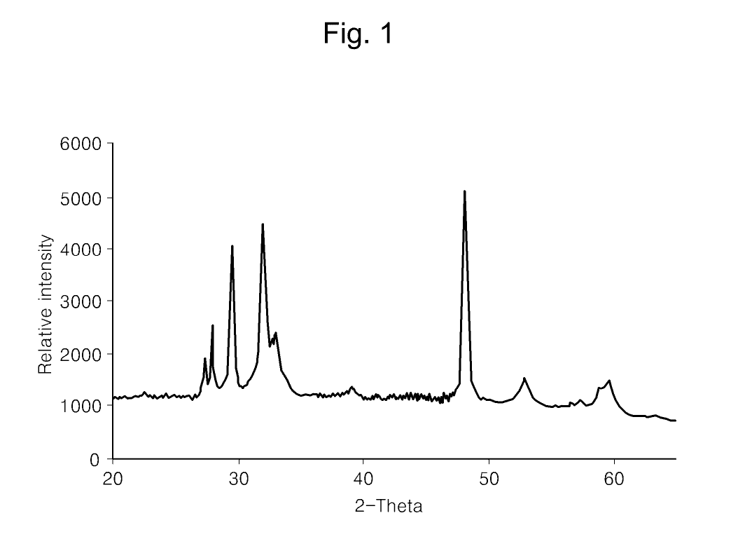

1 mole of CuSO₄ and 1 mole of Na₂S were added to distilled water and stirred for 30 minutes. The stirred mixture was added to an isothermal reactor at 50° C. and allowed to react for 30 minutes, thereby synthesizing fine copper sulfide particles. Herein, the sulfur content of the synthesized copper sulfide was 45 mole %. The synthesized copper sulfide had the characteristic crystal structure of copper sulfide as shown in FIG. 1. Referring to FIG. 1, a peak corresponding to the crystal structure of sulfur did not appear, because sulfur has no crystal structure. However, peaks corresponding to the crystal structure of copper appeared at 55°, 65°, 99°, 125° and 137°. The fine particles were observed using X-ray powder diffraction (XRD, XD-3A, Shimadzu, Japan). In order to coat the above-prepared copper sulfide on the surface of a biocompatible matrix made of a Ti alloy (Ti-6Al-4V), 0.1 wt % of the copper sulfide was added to isopropyl alcohol (IPA) and stirred at room temperature for 1 hour, thereby preparing a coating solution having excellent dispersibility. The coating solution was dip-coated on a Ti alloy biocompatible matrix having a diameter of 1 cm and a length of 10 cm. The coated Ti alloy was dried at 50° C. for 1 hour, and then annealed at 350° C. for 30 minutes. To provide a biomaterial having excellent antimicrobial activity, coating was repeated in the same manner as described above such that the copper sulfide could be sufficiently coated on the surface of the Ti alloy biocompatible material, thereby forming a coating layer having a thickness of 2 μm. The antimicrobial activity of the prepared biomaterial was measured as described above.

### Example 2

A coating solution containing 10 wt % of the copper sulfide synthesized as described in Example 1 was dip-coated on a biocompatible matrix made of hydroxyapatite and having a diameter of 1 cm and a length of 10 cm. The antimicrobial activity of the prepared biomaterial was measured as described above.

### Example 3

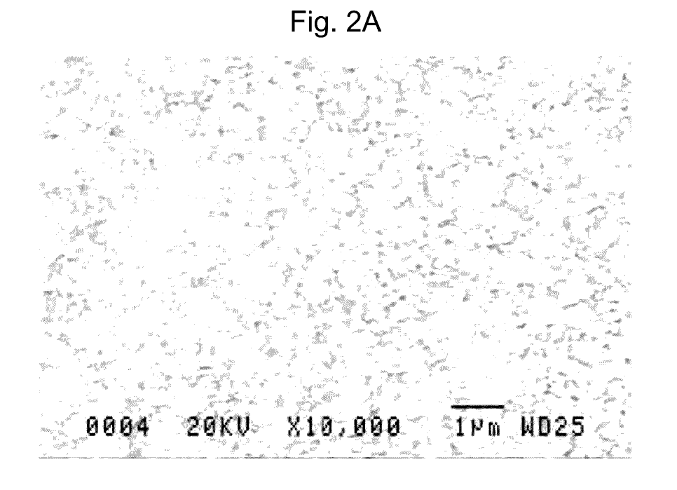

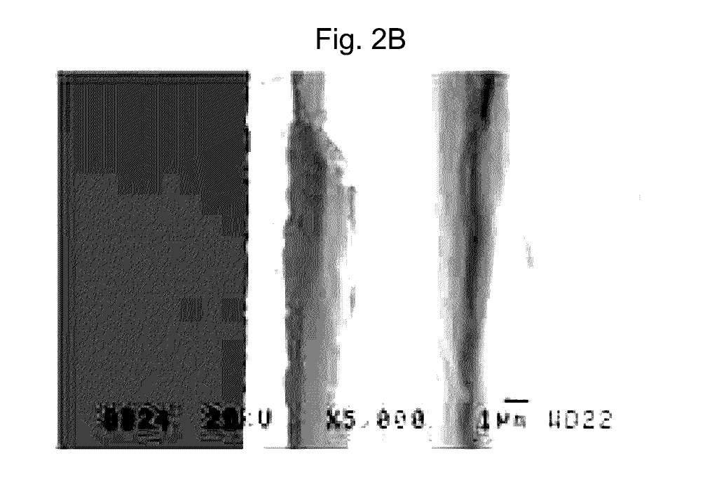

A coating solution containing 30 wt % of the copper sulfide synthesized as described in Example 1 was dip-coated on a biocompatible matrix made of low-density polyethylene (LDPE; specific gravity: 0.92) and having a diameter of 1 cm and a length of 10 cm. The antimicrobial activity of the prepared biomaterial was measured as described above. FIGS. 2A and 2B are top and cross-section photographs showing the copper sulfide coated on the biocompatible matrix made of LDPE.

### Example 4

0.1 wt % of the copper sulfide synthesized as described in Example 1 was added to low-density polyethylene (LDPE; specific gravity: 0.92), and 1.5 wt % of fine zinc (Zn) particles were added thereto in order to reduce extrusion pressure, after which a compounding process was performed to prepare chips. The prepared chips were injected using an injection molding machine at a temperature of 130° C. and an extrusion pressure of 0.07 kg/(mm²×h), thereby preparing an artificial biomaterial having a diameter of 1 cm and a length of 10 cm. Herein, a cooling process and an annealing process were performed to improve the mechanical properties of the biomaterial. The antimicrobial activity of the prepared biomaterial was measured as described above.

### Example 5

1 wt % of fine nickel particles and 10 wt % of the copper sulfide synthesized as described in Example 1 were added to low-density polyethylene. Using the mixture, an artificial biomaterial having a diameter of 1 cm and a length of 10 cm was prepared. Herein, the extrusion pressure was 0.1 kg/(mm²×h). The antimicrobial activity of the prepared biomaterial was measured as described above.

### Example 6

According to the same method as described in Example 4, 5 wt % of the copper sulfide and 0.2 wt % of manganese (Mn) were added to low-density polyethylene. Using the mixture, an artificial biomaterial having a diameter of 1 cm and a length of 10 cm was prepared. The antimicrobial activity of the prepared biomaterial was measured as described above.

### Example 7

According to the same method as described in Example 4, 20 wt % of the copper sulfide and 0.6 wt % of iron (Fe) were added to high-density polyethylene (HDPE). Using the mixture, an artificial biomaterial having a diameter of 1 cm and a length of 10 cm was prepared. The antimicrobial activity of the prepared biomaterial was measured as described above.

### Example 8

According to the same method as described in Example 4, 30 wt % of the copper sulfide and 0.7 wt % of cobalt was added to polypropylene (PP). Using the mixture, preparing an artificial biomaterial having a diameter of 1 cm and a length of 10 cm was prepared. Herein, the extrusion pressure was 0.3 kg/(mm²×h). The antimicrobial activity of the prepared biomaterial was measured as described above.

### Example 9

According to the same method as described in Example 4, 40 wt % of the copper sulfide and 2 wt % of chromium (Cr) were added to polypropylene terephthalate (PET). Using the mixture, an artificial biomaterial having a diameter of 1 cm and a length of 10 cm was prepared. Herein, the extrusion pressure was 0.5 kg/(mm²×h). The antimicrobial activity of the prepared biomaterial was measured as described above.

### Comparative Example 1

Using a Ti alloy (Ti-6Al-4V), an artificial biomaterial having a diameter of 1 cm and a length of 10 cm was prepared. The antimicrobial activity of the prepared biomaterial was measured as described above.

### Comparative Example 2

Using hydroxyapatite, an artificial biomaterial having a diameter of 1 cm and a length of 10 cm was prepared. The antimicrobial activity of the prepared biomaterial was measured as described above.

### Comparative Example 3

Using low-density polyethylene (LDPE), an artificial biomaterial having a diameter of 1 cm and a length of 10 cm was prepared. The antimicrobial activity of the prepared biomaterial was measured as described above.

### Comparative Example 4

According to the same method as described in Example 4, 20 wt % of copper sulfide and 0.01 wt % of iron (Fe) were added to high-density polyethylene (HDPE). Using the mixture, an artificial biomaterial having a diameter of 1 cm and a length of 10 cm was prepared. Herein, the extrusion pressure was 5 kg/(mm²×h). The antimicrobial activity of the prepared biomaterial was measured as described above.

### Comparative Example 5

According to the same method as described in Example 4, 30 wt % of copper sulfide and 40 wt % of cobalt (Co) were added to polypropylene (PP). Using the mixture, an artificial biomaterial having a diameter of 1 cm and a length of 10 cm was prepared. Herein, the extrusion pressure was 15 kg/(mm²×h). The antimicrobial activity of the prepared biomaterial was measured as described above.

### Comparative Example 6

According to the same method as described in Example 4, 40 wt % of copper sulfide and 2 wt % of aluminum (Al) were added to polyterephthalate (PET). Using the mixture, an artificial biomaterial having a diameter of 1 cm and a length of 10 cm was prepared. Herein, the extrusion pressure was 12 kg/(mm²×h). The antimicrobial activity of the prepared biomaterial was measured as described above.

Table 1 below compares the antimicrobial activity (cells/ml) of the artificial biomaterials prepared in Examples 1 to 9 of the present invention and Comparative Examples 1 to 6. ND in Table 1 means that the number of Escherichia coli (ATCC 25922) cells per ml cannot be measured, because it is more than 10¹⁰.

TABLE 1

| | | | Conductive particles | | | | |
|---|---|---|---|---|---|---|---|
| | | | Fine | Fine | | Container | |
| | | | copper | metal | | sheet | |
| | | | sulfide | particles | | | Anti- |
| | | | particles | Kind | Con- | Extrusion | microbial |
| | | Bio- | Content | of | tent | pressure | activity |
| | | material | (wt %) | metal | (wt %) | (ΔP/h) | (cells/mL) |
| Ex. | 1 | Ti alloy | 0.1 | / | / | / | 3.2 × 10⁴ |
| | 2 | Apatite | 10 | / | / | / | 3.4 × 10⁴ |
| | 3 | LDPE | 30 | / | / | / | 3.2 × 10⁴ |
| | 4 | LDPE | 0.1 | Zn | 1.5 | 0.07 | 4.0 × 10⁷ |
| | 5 | LDPE | 10 | Ni | 1 | 0.1 | 3.2 × 10⁶ |
| | 6 | LDPE | 5 | Mn | 0.2 | 0.05 | 6.5 × 10⁶ |
| | 7 | HDPE | 20 | Fe | 0.6 | 0.2 | 2.2 × 10⁵ |
| | 8 | PP | 30 | Co | 0.7 | 0.3 | 1.2 × 10⁵ |
| | 9 | PET | 40 | Cr | 2 | 0.5 | 1.3 × 10⁵ |
| Comp. | 1 | Ti alloy | / | / | / | / | ND |
| Ex. | 2 | Apatite | / | / | / | / | ND |
| | 3 | LDPE | / | / | / | / | ND |
| | 4 | LDPE | 20 | Fe | 0.01 | 5 | 7.2 × 10⁵ |
| | 5 | PP | 30 | Co | 40 | 15 | 5.2 × 10¹⁰ |
| | 6 | PET | 40 | Al | 2 | 12 | 6.2 × 10¹⁰ |

As shown in Table 1 above, in the case of the artificial biomaterials prepared by coating, the coating solution contained 0.1-30 wt % of copper sulfide particles having a particle size of about 55 nm. The artificial biomaterials of Examples 1 to 3 showed an antimicrobial activity of 3.2×10⁴ cells/mL. However, the artificial biomaterials of Comparative Examples 1 to 3, which were not coated with copper sulfide, showed very poor antimicrobial activity which could not be determined. It could be seen that the antimicrobial activity of the artificial biomaterial prepared by coating the copper sulfide was higher than those of the artificial biomaterials of Examples 4 to 9, prepared by dispersing the copper sulfide using the compounding method. However, the stability of the coating layer formed by the coating method can decrease with time, unlike that of the layer formed by the dispersion method. In the practical use of some artificial biomaterials, the stability of the coating layer needs to be considered.

In the case of the artificial biomaterials prepared by compounding, the biomaterials prepared in Examples 4 to 9 of the present invention had a copper sulfide content of 0.1-40 wt %. In addition, the fine metal particles added were fine particles of at least one selected from among chromium, manganese, iron, cobalt, nickel and zinc, and the concentration of the metal particles was 0.1-2 wt % based on the total weight of the biomaterial. In this case, the antimicrobial activity was 1.2×10⁵-6.5×10⁶ cells/mL. In addition, the extrusion pressure ranged from 0.05 to 0.5 kg/(mm²×h). However, the artificial biomaterials of Comparative Examples 1 to 3, which had no copper sulfide dispersed therein, showed very poor antimicrobial activity which could not be determined.

Comparative Example 4 corresponds to the case in which the concentration of fine iron (Fe) particles does not satisfy the condition of the example of the present invention (0.1-2 wt %; particle size: 10-30 nm), and Comparative Example 5 corresponds to the case in which the concentration of fine cobalt (Co) particles does not satisfy the condition of the example of the present invention. Herein, the antimicrobial activities of Comparative Examples 4 and 5 were 7.2×10⁵ cells/mL and 5.2×10¹⁰ cells/mL, respectively. Specifically, in the case of Comparative Example 4 in which the concentration of the fine metal particles was out of the range specified in the example of the present invention, the antimicrobial activity was not so poor, but the extrusion pressure was 5 kg/(mm²×h), which was unsuitable for extrusion. In addition, in the case of Comparative Example 5 in which the concentration of the fine metal particles was out of the range specified in the example of the present invention, the extrusion pressure was 15 kg/(mm²×h), indicating impossible extrusion, and the antimicrobial activity was also significantly poor.

Comparative Example 6 corresponds to the case in which the fine metal particles added are not chromium, manganese, iron, cobalt, nickel or zinc particles, which are used in the present invention, but are aluminum (Al) particles. In this case, the antimicrobial activity was 6.2×10¹⁰ cells/mL, and the extrusion pressure was 12 kg/(mm²×h). Aluminum is a typical metal belonging to period 3 of the periodic table. It differs from the transition metal in period 4 of the periodic table, which is used in the present invention. When aluminum is added, the antimicrobial activity is poor, and the extrusion pressure is also high, resulting in a decrease in production efficiency. For this reason, the fine metal particles that are used in the present invention are preferably fine particles of a metal selected from among chromium, manganese, iron, cobalt, nickel and zinc, which are transition metals belonging to period 4 of the periodic table.

Artificial Joint Plated with Copper Sulfide

An artificial joint consists of two joint components made of a metal or ceramic material, and can substitute for the function of the joint of the human body. The artificial joint can rotate with the motion of the user or resist the load of the user. Although various materials may be used for the artificial joints, high-hardness metals such as Ti alloy steel or ceramic materials are frequently used, which resist the load of the user and have strong abrasion resistance. Such artificial joints are prepared in an elaborate form including various bent portions. The surface of the artificial joint is made smooth in order to minimize the frictional force caused by motion.

The plating method has disadvantages in that it is difficult to carry out and is expensive, compared to the deposition or wet application method. However, it is suitable for the preparation of an artificial joint that is repeatedly used over a long period of time because of its excellent durability. To increase the strength of the plating layer, the surface of the artificial joint is preferably treated with a conductive polymer emulsion solution containing a transition metal, because it is plated. To the surface of the artificial joint, an aqueous dispersion containing 0.01-1.0 wt % of colloidal transition metal particles and 0.01-2.0 wt % of an emulsion of at least one selected from water-soluble polyester, water-soluble urethane and water-soluble acrylic resin is applied. The solid content of the aqueous dispersion is controlled such that the solids of the aqueous dispersion remain on the surface of the artificial joint in an amount of 0.001-0.1 g/m². Although the plating process may also be performed by introducing copper sulfide into a plating bath, ionizing the introduced copper sulfide, and then plating the copper sulfide on a biocompatible material, a method is more preferably used in which a copper salt and a sulfur compound are added to a plating bath and copper sulfide is deposited using a reducing agent so that it is plated on the surface of a biocompatible salt. Copper sulfide is preferably plated on the biocompatible material to a thickness of 0.01-5.0 μm.

Hereinafter, the present invention will be described in further detail with reference to the following examples. It is to be understood, however, that these examples are for illustrative purposes only and are not intended to limit the scope of the present invention. The performance of artificial joints prepared in the examples of the present invention and comparative examples was evaluated as described below. The following description will be made with a focus on copper sulfide among the above-described compounds.

(1) Surface State of Coating Layer

In order to evaluate whether or not the surface of the coating layer has irregularities and to evaluate the plating state of the coating layer, the surface state of the coating layer was observed with a microscope, and the number of irregularities and non-plated portions per area of 10 cm×10 cm was observed and evaluated. The results of the evaluation were rated on the following scale:

∘: 1 or less;

Δ: 2 to 4;

x: 5 or more.

(2) Thickness of Coating Layer

The thickness of the coating layer was measured by SEM (scanning electron microscopy, JSM-6390A, JEOL, USA).

(3) Strength of Coating Layer

The adhesive strength of the coating layer of the coating layer was measured using a pull-down breaking point tester (Photo Technica, USA) while the coating layer was pulled with a load of 90 kg/sec. The results of the measurement were rated on the following scale:

∘: 700 kg/cm² or more;

Δ: 100-700 kg/cm²;

x: 100 kg/cm² or less.

(4) Antimicrobial Activity

Escherichia coli (ATCC 25922) was used as a test strain. A sample was brought into contact with the test strain, and then incubated at 25° C. for 24 hours. Then, the number of the bacterial cells was counted, thereby evaluating the antimicrobial activity of the sample. The antimicrobial activity of the sample was rated on the following scale: @=excellent (the number of Escherichia coli (ATCC 25922) cells was less than 10⁷); ND=the number of Escherichia coli (ATCC 25922) cells per mL was more than 10¹⁰).

### Example 10

An aqueous dispersion containing 0.5 wt % of fine colloidal particles of a transition metal and 1.0 wt % of a water-soluble polyester emulsion was applied to the surface of a Ti alloy (Ti-6Al-4V). The solid content of the aqueous dispersion was controlled such that the solids of the aqueous dispersion would remain on the surface of the Ti alloy in an amount of 0.05 g/m². Next, CuSO₄ and Na₂S were added to a plating solution at a molar ratio of 1:1, and copper sulfide was deposited on the surface of a Ti alloy (Ti-6Al-4V) having a diameter of 1 cm and a length of 10 cm by use of a reducing agent. The thickness of the plated copper sulfide was 2 μm.

As shown in FIG. 1, the crystal structure of the deposited copper sulfide had the characteristic structure of copper sulfide. Referring to FIG. 1, a peak corresponding to the crystal structure of sulfur did not appear, because sulfur has no crystal structure. However, peaks corresponding to the crystal structure of copper appeared at 55°, 65°, 99°, 125° and 137°. Observation of the fine copper sulfide particles was performed using X-ray powder diffraction (XRD, XD-3A, Shimadzu, Japan). The antimicrobial activity, surface state and coating layer strength of the artificial joint prepared as described above were measured according to the above-described methods.

### Example 11

As described in Example 10, copper sulfide was deposited on a Cr—Co—Mo alloy (SCM440H) having a diameter of 1 cm and a length of 10 cm to form a coating layer. The antimicrobial activity, surface state and coating layer strength of the artificial joint prepared as described above were measured according to the above-described methods.

### Example 12

As described in Example 10, copper sulfide was deposited on a zirconia-alumina composite ceramic having a diameter of 1 cm and a length of 10 cm to form a coating layer. The antimicrobial activity, surface state and coating layer strength of the artificial joint prepared as described above were measured according to the above-described methods.

### Comparative Example 7

0.1 wt % of copper sulfide was added to isopropyl alcohol (IPA) and stirred at room temperature for 1 hour to prepare a coating solution. The coating solution was dip-coated on a Ti alloy (Ti-6Al-4V) having a diameter of 1 cm and a length of 10 cm. The coated Ti alloy (Ti-6Al-4V) was dried at 50° C. for 1 hour, and then annealed at 400° C. for 30 minutes. To provide an artificial joint having excellent antimicrobial activity, coating was repeated in the same manner as described above so that copper sulfide could be sufficiently coated on the surface of the Ti alloy (Ti-6Al-4V), thereby forming a coating layer having a thickness of 2 μm. The antimicrobial activity, surface state and coating layer strength of the artificial joint prepared as described above were measured according to the above-described methods.

### Comparative Example 8

Copper sulfide was synthesized to prepare a vacuum deposition target. An aqueous dispersion containing 0.5 wt % of fine colloidal particles of a transition metal and 1.0 wt % of a water-soluble polyester emulsion was applied to the surface of a Ti alloy (Ti-6Al-4V) having a diameter of 1 cm and a length of 10 cm. The solid content of the aqueous dispersion was controlled such that the solids of the aqueous dispersion would remain on the surface of the Ti alloy in an amount of 0.05 g/m². For vapor deposition, heating was performed under a vacuum of 10⁻⁵-10⁻³ Torr such that the vapor pressure of the metal (10⁻²-10⁻¹ Torr) would be maintained, thereby depositing copper sulfide on the surface of the Ti alloy (Ti-6Al-4V) to a thickness of 400 Å. The antimicrobial activity, surface state and coating layer strength of the artificial joint prepared as described above were measured according to the above-described methods.

### Comparative Example 9

The antimicrobial activity and coating layer strength of a Ti alloy (Ti-6Al-4V) having a diameter of 1 cm and a length of 10 cm, on which a copper sulfide coating layer was not formed, were measured according to the above-described methods.

Table 2 below compares the antimicrobial activity (cells/mL), surface state (irregularities/100 cm²) and coating layer strength (kg/cm²) of the artificial joints prepared in Examples 10 to 12 of the present invention and Comparative Examples 7 to 9.

TABLE 2

| | | | | | Properties | | |
|---|---|---|---|---|---|---|---|
| | | | Presence | | | | Coat- |
| | | | of | | Anti- | Sur- | ing |
| | | Artificial | copper | Coating | microbial | face | layer |
| | | joint | sulfide | method | activity | state | strength |
| Ex. | 10 | Ti alloy | ∘ | Plating | ∘ | ∘ | ∘ |
| | 11 | Co alloy | ∘ | Plating | ∘ | ∘ | ∘ |
| | 12 | Zirconia- | ∘ | Plating | ∘ | ∘ | ∘ |
| | | based | | | | | |
| | | ceramic | | | | | |
| Comp. | 7 | Ti alloy | ∘ | Wet | ∘ | x | x |
| Ex. | | | | application | | | |
| | 8 | Ti alloy | ∘ | Deposition | ∘ | Δ | Δ |
| | 9 | Ti alloy | x | / | ND | / | / |

As can be seen in Table 2 above, the artificial joints of Examples 10 to 12 and Comparative Example 7 or 8, prepared by forming the copper sulfide coating layer using the plating, wet application or deposition method, showed excellent antimicrobial activity. This suggests that the copper sulfide coating layer shows high antimicrobial activity regardless of the coating method. In comparison with this, the artificial joint of Comparative Example 9, which has no copper sulfide coating layer, showed very poor antimicrobial activity that could not be determined. However, the coating layer formed by the wet application method, not the plating method, showed a strength of 100 kg/cm² or lower, and the coating layer formed by the deposition method showed a strength lower than 700 kg/cm² but higher than 100 kg/cm². A coating layer having a strength lower than 700 kg/cm² is difficult to apply to the artificial joint according to the embodiment of the present invention. In addition, the surface state of the coating layer formed by the plating method was better than that of the coating layer formed by the wet application method or the deposition method. Thus, it could be seen that the coating method suitable for the preparation of the artificial joints was the plating method.

As described above, the artificial biomaterial comprising the copper-based compound according to the present invention has coated thereon or dispersed therein the copper sulfide compound, and thus is relatively low-priced, is easy to process and is non-toxic. Moreover, because the copper sulfide compound has excellent antimicrobial activity, the application of this compound to an artificial joint can increase the antimicrobial activity of the artificial joint. In addition, the plating layer formed by plating the copper sulfide compound on the surface of an artificial joint that is an artificial biomaterial has a smooth surface consistent with the shape of the artificial joint and shows high antimicrobial activity and high layer strength. Furthermore, because the plating layer on the artificial joint has relatively high layer strength, the plating method is a coating method suitable for the artificial joint that rotates repeatedly and that should resist the load of the user.

Although the preferred embodiments of the present invention have been described in detail, the scope of the present invention is not limited to these embodiments, and those skilled in the art will appreciate that various modifications are possible, without departing from the scope and spirit of the invention as disclosed in the accompanying claims.

1.-20. (canceled)

21. An artificial biomaterial comprising a copper-based compound, the artificial biomaterial comprising:
a biocompatible matrix suitable for insertion into the human body; and
either a coating layer comprising the copper-based compound, formed on the surface of the matrix, or the copper-based compound dispersed in the matrix to form a bulk material.
wherein the copper-based compound has a chemical structure of CuₓM_{y}, wherein M is any one selected from among elements belonging to groups 15 to 17 of the periodic table, and x/y=0.8-1.5.

22. The artificial biomaterial of claim 21, wherein M is any one selected from among S, F and Cl.

23. The artificial biomaterial of claim 21, wherein the compound is copper sulfide.

24. The artificial biomaterial of claim 21, wherein the compound is contained in an amount of more than 0 wt % but not more than 50 wt % based on the weight of the bulk material.

25. The artificial biomaterial of claim 21, wherein the compound is contained in an amount of 0.01-30 wt % based on the weight of the bulk material.

26. The artificial biomaterial of claim 21, wherein the coating layer is formed by any one method selected from among wet application, vapor deposition, and coating.

27. The artificial biomaterial of claim 26, wherein the coating layer is formed by wet application, and a coating solution for forming the coating layer contain the compound in an amount of more than 0 wt % but not more than 50 wt %.

28. The artificial biomaterial of claim 26, wherein the coating layer is formed by wet application, and a coating solution for forming the coating layer contains the compound in an amount of 0.01-30 wt %.

29. The artificial biomaterial of claim 21, wherein the artificial biomaterial comprises the matrix made of any one selected from among a metal material, a ceramic material and a composite material comprising at least one of these materials.

30. The artificial biomaterial of claim 29, wherein the artificial biomaterial comprises the compound coated on the surface of the matrix made of any one selected from among a metal material, a ceramic material and a composite material comprising at least one of these materials.

31. The artificial biomaterial of claim 29, wherein the artificial biomaterial comprises the compound coated on the surface of the matrix made of any one selected from among a polymer material and a composite material comprising the polymer material, comprises the compound dispersed in the matrix.

32. The artificial biomaterial of claim 21, wherein the artificial biomaterial comprising the compound dispersed therein comprises fine particles of at least one metal selected from among chromium, manganese, iron, cobalt, nickel and zinc in an amount of 0.1-2 wt % based on the total weight of the biomaterial.

33. The artificial biomaterial of claim 21, wherein the artificial biomaterial is applied to an artificial joint, and comprises a layer of the compound plated on the surface of a matrix for the artificial joint.

34. The artificial biomaterial of claim 33, wherein the plating is electroless plating or electroplating.

35. The artificial biomaterial of claim 33, wherein the plating is performed by performing electroless plating followed by electroplating.

36. The artificial biomaterial of claim 33, wherein the plating layer has a thickness of 0.01-5.0 μm.

37. The artificial biomaterial of claim 33, wherein the surface of the artificial joint is treated with a conductive polymer emulsion solution containing a transition metal, before the plating layer is formed.

38. The artificial biomaterial of claim 37, wherein an aqueous dispersion containing 0.01-1.0 wt % of fine colloidal particles of a transition metal and 0.01-2.0 wt % of an emulsion of at least one selected from water-soluble polyester, water-soluble urethane and water-soluble acrylic resin is applied to the surface of the artificial joint.

39. The artificial biomaterial of claim 38, wherein the solid content of the aqueous dispersion is controlled such that solids of the aqueous dispersion remain on the surface of the artificial joint in an amount of 0.001-0.1 g/m².

40. The artificial biomaterial of claim 21, wherein the biocompatible matrix is made of polyurethane resin or silicone resin.
